(19) United States
(12) Patent Application Publication
(10) Pub. No.: US 2003/0145393 A1
(43) Pub. Date: Aug. 7, 2003

(54) Hair dyes containing cationic biopolymers

(76) Inventors: Alberto Corbella, Como (IT); Giovanni Guidotti, Cadorago (IT)

Correspondence Address: COGNIS CORPORATION, 2500 RENAISSANCE BLVD., SUITE 200, GULPH MILLS, PA, 19406

(21) Appl. No.: 10/221,959

(22) PCT Filed: Mar. 15, 2001
(86) PCT No.: PCT/EP01/02937

(30) Foreign Application Priority Data
Mar. 24, 2000 (DE) 10014340.7

(51) Int. Cl. A61K 7/13

(52) U.S. Cl. 8/405; 8/407

(57) ABSTRACT

The invention relates to hair dyes comprising good properties with regard to dyeing technology and hair care, which are based on cationic biopolymers, alkyl and/or alkenyl oligoglycosides and colorants, and to their use for dying keratin fibers.

### FIELD OF THE INVENTION

The invention is in the field of hair cosmetics and relates to hair dyes based on cationic biopolymers, alkyl and/or alkenyl oligoglycosides and dyes, and to their use for dyeing keratin fibers.

### PRIOR ART

Conventional hair dyes can be divided into three different groups, direct temporary, semipermanent and permanent preparations.

Temporary, direct hair dyes are characterized by a temporary change in the existing hair color, which is achieved by depositing the dyes on the surface of the hair. This deposition can be readily washed off or out again by washing the hair. Application forms which are often available are aqueous-alcoholic lotions or foam aerosols. By using semipermanent hair dyes, by using nonionic or cationic dyes with high affinity to the keratin and good penetrability, a coloration is achieved which can be washed out in a delayed manner over 5-6 hair washes. The dyes are used in a shampoo, soap or cream base.

Permanent hair dyes which dye the hair permanently in most cases contain oxidation dyes. Available formulations are creams, gels or shampoos.

Depending on the hair-dyeing method, undesired damage to the hair structure may result. This damage is evident inter alia from poor wet and dry combability, increased electrostatic charging, increased brittleness, reduced maximum breaking force and tear elongation of the hair, and a poorer external appearance of the hairstyle. Furthermore, dyes may, for example, in some cases lead to uneven dyeing which is even visible to the naked eye, or in the case of semipermanent colorations only a low intensity may be achieved, which rapidly fades.

There has therefore increasingly been a search for formulations which overcome these disadvantages.

To improve the dyeing properties, the use of alkyl oligoglycosides as dispersant in dyes has proven advantageous. It is generally praised for the preparation of dye and pigment preparations in the patent specification EP 658 165 B1. In the field of hair cosmetics, it is the subject-matter of the patent specification DE 41 29 926 C1, which discloses hair tints with gentle cleaning and improved color substantivity, which comprise a direct dye in a surfactant base with 5-35% by weight of an anion-active surfactant, 0.5-10% by weight of a surface-active amine oxide and 2.5-15% by weight of an alkyl oligoglycoside. Hair dyes containing alkyl oligoglycosides and oxidation dyes is also given in the patent specification EP 655 905 B1, the disclosed compositions are characterized by good dyeing properties and good combability of the treated hair.

An improved hair-conditioning action, such as feel, shine and combability is also achieved through use of water-soluble chitosan salts as constituent in hair cures and hair shampoos. The corresponding formulations may here also comprise direct dyes and quaternary ammonium salts (cf. DE 2754796 C).

The patent specification EP 137 178 A discloses hair-tinting shampoos with direct dyes and, in addition to an anionic surfactant, a so-called auxiliary surfactant, which should be a cationic and/or betaine surfactant.

The object of the invention was consequently to provide a formulation which counteracts the disadvantageous effects of dyes on the hair. This formulation should on the one hand have good dyeing properties, lead to good color distribution and intensity, and also ensure a longer-lasting tinting in the case of semipermanent colorations. On the other hand, it should have good care properties and be characterized by good biological compatibility. An improved hair-conditioning effect, such as good wet and dry combability, reduced electro-static charging, increased strength and tear elongation are further aims of the object.

### DESCRIPTION OF THE INVENTION

The invention provides hair dyes which contain cationic biopolymers, alkyl and/or alkenyl oligoglycosides and dyes. The invention further provides for the use of mixtures which comprise cationic biopolymers, alkyl and/or alkenyl oligoglycosides and dyes for dyeing keratin fibers.

Surprisingly, it has been found that hair dyes with a composition containing cationic biopolymers, alkyl and/or alkenyl oligoglycosides and dyes have improved dyeing and hair-conditioning effects compared with conventional formulations.

While the use of alkyl oligoglycosides leads to good dispersibility and thus color distribution, the cationic biopolymers bring about, by forming a fine film on the hair, better adhesion of the dye and thus a more intense and longer-lasting color effect. As a result of the film formation, the setting of the hair is increased, the electrostatic charging is reduced and the surface is smoother and can thus be combed more easily. The elasticity of the film protects the hair against breakage. In particular, the combination of alkyl and/or alkenyl oligoglycosides and cationic biopolymers leads, despite improved dyeing effect, to particularly gentle hair dyes and to an additional hair-setting action.

Both alkyl and/or alkenyl oligoglycosides and also cationic biopolymers are characterized by good biological compatibility and biodegradability.

Cationic Biopolymers

The best known, and for the purposes of the invention also most preferred, cationic biopolymers include the chitosans, which also belong to the group of hydrocolloids. From a chemical viewpoint, they are partially deacetylated chitins of varying molecular weight which contain the following—idealized—monomer building block:

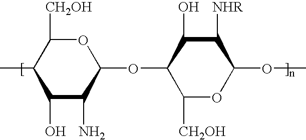

The chitosans are prepared starting from chitin, preferably the shell remains of crustaceans, which are available as inexpensive raw materials in large amounts. Here, the chitin is usually, in a process which was described for the first time by Hackmann et al., firstly deproteinated by the addition of bases, demineralized by the addition of mineral acids and, finally, deacetylated by the addition of strong bases, it being possible for the molecular weight to be distributed over a broad spectrum. Corresponding processes are known, for example, from Makromol. Chem. 177, 3589 (1976) or the French patent application FR 2701266 A1. Use is usually made of chitosans with an average molecular weight of from 10 000 to 5 000 000 daltons, in a preferred embodiment chitosans with an average molecular weight of from 30 000 to 100 000 daltons are used, and chitosans with a molecular weight of from 100 000 to 1 000 000 daltons are also preferred. Particular preference is given to using those grades disclosed in German patent applications DE 4442987 A1 and 19537001 A1 (Henkel) and which have an average molecular weight of from 800 000 to 1 200 000 daltons, a Brookfield viscosity (1% strength by weight in glycolic acid) of less than 5000 mPas, a degree of deacetylation in the range from 80 to 88% and an ash content of less than 0.3% by weight.

In addition to the chitosans as typical cationic biopolymers, for the purposes of the invention, anionically, nonionically or cationically derivatized chitosans, such as, for example, carboxylation, succinylation, alkoxylation or quaternization products, are suitable, as are described, for example, in German patent specification DE 3713099 C2, and German patent application DE 19604180 A1.

In addition, the generic term cationic biopolymers should also be understood as including related feed substances, such as, for example, gelatins, collagens and collagen degradation products.

Alkyl and/or Alkenyl Oligoglycosides

Alkyl and alkenyl oligoglycosides which are suitable as emulsifier component are known nonionic surfactants which conform to the formula (I),

R¹O—[G]ₚ (I)

in which R¹ is an alkyl and/or alkenyl radical having 4 to 22 carbon atoms, G is a sugar radical having 5 or 6 carbon atoms and p is a number from 1 to 10. They can be obtained by the relevant methods of preparative organic chemistry. By way of representatives for the extensive literature, reference is made here to the specifications EP-A1 0 301 298 and WO 90/03977.

The alkyl and/or alkenyl oligoglycosides can be derived from aldoses or ketoses having 5 or 6 carbon atoms, preferably glucose. The index number p in the general formula (I) gives the degree of oligomerization (DP), i.e. the distribution of mono- and oligoglycosides and is a number between 1 and 10. While p in a given compound must always be an integer and here primarily may assume the values p=1 to 6, the value p for a certain alkyl oligoglycoside is an analytically determined parameter which in most cases is a fraction. Preference is given to using alkyl and/or alkenyl oligoglycosides with an average degree of oligomerization p of from 1.1 to 3.0. From a performance viewpoint, preference is given to those alkyl and/or alkenyl oligoglycosides whose degree of oligomerization is less than 1.7 and is in particular between 1.2 and 1.4.

The alkyl or alkenyl radical R¹ can be derived from primary alcohols having 4 to 11, preferably 8 to 10, carbon atoms. Typical examples are butanol, caproic alcohol, caprylic alcohol, capric alcohol and undecyl alcohol, and technical-grade mixtures thereof, as are obtained, for example, in the hydrogenation of technical-grade fatty acid methyl esters or in the course of the hydrogenation of aldehydes from the Roelen oxo synthesis. Preference is given to alkyl oligoglucosides of chain length C₈-C₁₀ (DP=1 to 3), which are produced as forerunnings in the distillative separation of technical-grade C₈-C₁₈-coconut fatty alcohol and may be contaminated with a fraction of less than 6% by weight m/m of C₁₂-alcohol, and alkyl oligoglucosides based on technical-grade C_{9/11}-oxo alcohols (DP=1 to 3). The alkyl or alkenyl radical R¹ can also be derived from primary alcohols having 12 to 22, preferably 12 to 14, carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol, and technical-grade mixtures thereof which may be obtained as described above. Preference is given to alkyl oligoglucosides based on hydrogenated C_{12/14}-cocoalcohol with a DP of from 1 to 3.

Dyes

To dye keratin fibers, preferably human hair, either so-called direct dyes or oxidation dyes are usually used.

The latter are composed of a developer component (oxidation base) and a coupler component (nuancer) and do not represent dyes in the actual sense, but dye precursors.

Oxidation bases are aromatic compounds which are ring-substituted by at least two electron-releasing groups (amino and/or hydroxyl groups). Use is made, for example, of primary aromatic amines with a further free or substituted hydroxyl or amino group in the para or ortho position, diaminopyridine derivatives, hetero-cyclic hydrazones, 4-aminopyrazolone derivatives, and 2,4,5,6-tetraaminopyrimidine and derivatives thereof. Specific representatives are, inter alia, p-tolylenediamine, p-aminophenol, N,N-bis(2-hydroxyethyl)-p-phenylenediamine, 2-(2,5-diaminophenoxy)ethanol, 1-phenyl-3-carboxyamido-4-amino-5-pyrazolone and 4-amino-3-methylphenol, 2-(2-hydroxyethyl)-1,4-amino-benzene and 2,4,5,6-tetraaminopyrimidine.

Nuancers are generally likewise aromatic compounds, but with readily oxidizable groups on the ring in the m-position. Components which are available are usually m-phenylenediamine derivatives, naphthols, resorcinol and resorcinol derivatives, pyrazolones, m-amino-phenols, and pyridine derivatives. Suitable coupler substances are, in particular, 1-naphthol, pyrogallol, 1,5-, 2,7- and 1,7-dihydroxynaphthalene, 5-amino-2-methylphenol, m-aminophenol, resorcinol, resorcinol monomethyl ether, m-phenylenediamine, 1-phenyl-3-methyl-5-pyrazolone, 2,4-dichloro-3-aminophenol, 1,3-bis(2,4-diaminophenoxy)propane, 2-chlororesorcinol, 2-chloro-6-methyl-3-aminophenol, 2-methylresorcinol, 2,5-dimethylresorcinol, 2,6-dihydroxy-pyridine and 2,6-diaminopyridine.

Suitable direct dyes are, for example, dyes from the group of nitrophenylenediamines, nitroaminophenols, anthraquinones or indophenols, such as, for example, the compounds known under the international names or trade names HC Yellow 2, HC Yellow 4, Basic Yellow 57, Disperse Orange 3, HC Red 3, HC Red BN, Basic Red 76, HC Blue 2, Disperse Blue 3, Basic Blue 99, HC Violet 1, Disperse Violet 1, Disperse Violet 4, Disperse Black 9, Basic Brown 16, Basic Brown 17, picramic acid and Rodol 9 R, and 4-amino-2-nitrodiphenylamine-2′-carboxylic acid, 6-nitro-1,2,3,4-tetrahydroquinoxaline, (N-2,3-dihydroxypropyl-2-nitro-4-trifluoromethyl)aminobenzene and 4-N-ethyl-1,4-bis(2′-hydroxyethylamino)-2-nitro-benzene hydrochloride.

In addition to synthetic fibers, naturally occurring dyes may also be used, such as, for example, henna red, henna neutral, henna black, camomile blossom, sandalwood, black tea, buckthorn bark, sage, logwood, madder root, catechu, cedar, alkanna root, turmeric, hematoxylin and autin. The natural dyes cannot be clearly assigned to the two groups, but in the most frequent cases fall into the group of direct dyes.

In addition to dye mixtures within the groups, mixtures of dyes from different groups can also be used.

With regard to further dye components, reference is expressly made to the Colipa list, published by the Industrieverband Körperpflege und Waschmittel, Frankfurt. An overview of suitable dyes is also given in the publication “Kosmetische Färbemittel” [Cosmetic dyes] by the Dyes Commission of the German Research Society, Verlag Chemie, Weinheim, 1984, pp. 81-106.

Cationic Surfactants

Examples of the cationic surfactants which can optionally be used in the hair-treatment compositions according to the invention are, in particular, quaternary ammonium compounds. Preference is given to ammonium halides, such as alkyltrimethylammonium chlorides, dialkyldimethylammonium chlorides and trialkylmethylammonium chlorides, e.g. cetyltrimethylammonium chloride, stearyltrimethylammonium chloride, distearyldimethylammonium chloride, lauryldimethylammonium chloride, lauryldimethylbenzylammonium chloride and tricetylmethylammonium chloride.

Quaternary ester compounds, so-called “ester quats” are likewise very biodegradable.

The term “ester quats” is generally to be understood as meaning quaternized fatty acid triethanolamine ester salts. These are known substances which can be obtained by the relevant methods of preparative organic chemistry. In this connection, reference may be made to the international patent application WO 91/01295 (Henkel), which involves partially esterifying triethanolamine in the presence of hypophosphorus acid with fatty acids, passing air through and then quaternizing with dimethyl sulfate or ethylene oxide. Moreover, German patent specification DE 4308794 C1 (Henkel) discloses a process for the preparation of solid ester quats in which the quaternization of triethanolamine esters is carried out in the presence of suitable dispersants, preferably fatty alcohols. Overviews on this topic are given, for example, by R. Puchta et al. in Tens. Surf. Det., 30, 186 (1993), M. Brock in Tens. Surf. Det., 30, 394 (1993), R. Lagerman et al. in J. Am. Oil. Chem. Soc., 71, 97 (1994) and I. Shapiro in Cosm. Toil. 109, 77 (1994).

The quaternized fatty acid triethanolamine ester salts conform to the formula (II),

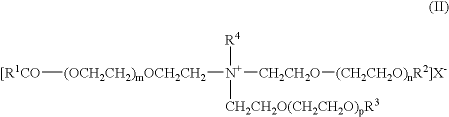

in which R¹CO is an acyl radical having 6 to 22 carbon atoms, R² and R³, independently of one another, are hydrogen or R¹CO, R⁴ is an alkyl radical having 1 to 4 carbon atoms or a (CH₂CH₂O)_{q}H group, m, n and p are in total 0 or numbers from 1 to 12, q is a number from 1 to 12 and X is halide, alkylsulfate or alkylphosphate. Typical examples of ester quats which can be used for the purposes of the invention are products based on caproic acid, caprylic acid, capric acid, lauric acid, myristic acid, palmitic acid, isostearic acid, stearic acid, oleic acid, elaidic acid, arachidic acid, behenic acid and erucic acid, and technical-grade mixtures thereof, as are produced, for example, in the pressurized cleavage of natural fats and oils. Preference is given to using technical-grade C_{12/18}-coconut fatty acids and, in particular, partially hydrogenated C_{16/18}-tallow or palm fatty acids, and elaidic acid-rich C_{16/18}-fatty acid cuts. To prepare the quaternized esters, the fatty acids and the triethanolamine can be used in the molar ratio from 1.1:1 to 3:1. With regard to the performance properties of the ester quats, a feed ratio of 1.2:1 to 2.2:1, preferably 1.5:1 to 1.9:1, has proven particularly advantageous. The preferred ester quats are technical-grade mixtures of mono-, di- and triesters with an average degree of esterification of from 1.5 to 1.9 and are derived from technical-grade C_{16/18}-tallow or palm fatty acid (iodine number 0 to 40). From a performance viewpoint, quaternized fatty acid triethanolamine ester salts of the formula (I) in which R¹CO is an acyl radical having 16 to 18 carbon atoms, R² is R¹CO, R³ is hydrogen, R⁴ is a methyl group, m, n and p are 0 and X is methylsulfate have proven particularly advantageous. Corresponding products are available commercially under the name Dehyquart® AU (Cognis Deutschland GmbH).

In addition to the quaternized fatty acid triethanolamine ester salts, suitable ester quats are also quaternized ester salts of fatty acids with diethanolalkylamines of the formula (III),

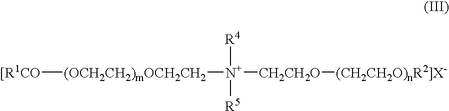

in which R¹CO is an acyl radical having 6 to 22 carbon atoms, R² is hydrogen or R¹CO, R⁴ and R⁵, independently of one another, are alkyl radicals having 1 to 4 carbon atoms, m and n are in total 0 or numbers from 1 to 12 and X is halide, alkylsulfate or alkylphosphate.

Finally, a further group of suitable ester quats are the quaternized ester salts of fatty acids with 1,2-dihydroxypropyldialkylamines of the formula (IV)

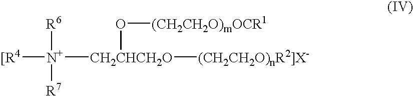

in which R¹CO is an acyl radical having 6 to 22 carbon atoms, R² is hydrogen or R¹CO, R⁴, R⁶ and R⁷, independently of one another, are alkyl radicals having 1 to 4 carbon atoms, m and n are in total 0 or numbers from 1 to 12 and X is halide, alkylsulfate or alkylphosphate.

In addition, suitable ester quats are also substances in which the ester bond is replaced by an amide bond and which preferably conform on the basis of diethylenetriamine to the formula (V),

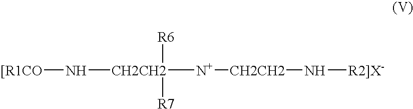

in which R1CO is an acyl radical having 6 to 22 carbon atoms, R2 is hydrogen or R1CO, R6 and R7, independently of one another, are alkyl radicals having 1 to 4 carbon atoms and X is halide, alkylsulfate or alkylphosphate. Such amide ester quats are available commercially, for example, under the name Incroquat® (Croda).

Finally, suitable ester quats are also substances which are obtainable on the basis of ethoxylated castor oil and its hydrogenation products and preferably conform to the formula (VI),

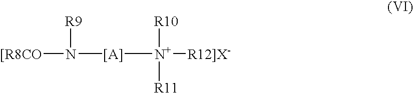

in which R8CO is a saturated and/or unsaturated ethoxylated hydroxyacyl radical having 16 to 22, preferably 18, carbon atoms, and 1 to 50 oxyethylene units, A is a linear or branched alkylene radical having 1 to 6 carbon atoms, R9, R10 and R11, independently of one another, are hydrogen or an alkyl group having 1 to 4 carbon atoms, R12 is an alkyl radical having 1 to 4 carbon atoms or a benzyl radical and X is halogen, alkylsulfate or alkylphosphate.

With regard to the choice of preferred fatty acids and the optimum degree of esterification, the examples given for (I) also apply to the ester quats of the formulae (III) to (VI).

The ester quats of the formulae (II) to (VI) can be prepared starting either from fatty acids or the corresponding triglycerides. Such a process, which is to be specified as being representative of the corresponding prior art, is proposed in European patent specification EP 0750606 B1 (Cognis). It is likewise possible to carry out the condensation of the alkanolamines with the fatty acids in the presence of defined amounts of dicarboxylic acids, such as, for example, oxalic acid, malonic acid, succinic acid, maleic acid, fumaric acid, glutaric acid, adipic acid, sorbic acid, pimelic acid, azelaic acid, sebacic acid and/or dodecanedioic acid. This method results in a partially oligomeric structure of the ester quats, which, particularly when co-used with adipic acid, can have an advantageous effect on the ability of the products to form clear solutions. Corresponding products under the name Dehyquart® D 6003 (Cognis Deutschland GmbH) are commercially available and are described, for example, in European patent specification EP 0770594 B1 (Cognis). The ester quats are usually supplied commercially in the form of 50 to 90% strength by weight alcoholic solutions which can be diluted with water without problems as required.

Likewise suitable according to the invention are cationic silicone oils, such as, for example, the commercially available products Q2-7224 (manufacturer: Dow Corning; a stabilized trimethylsilylamodimethicone), Dow Corning 929 Emulsion (comprising a hydroxyamino-modified silicone, which is also referred to as amodimethicone), SM-2059 (manufacturer: General Electric), SLM-55067 (manufacturer: Wacker) and Abil®-Quat 3270 and 3272 (manufacturer: Th. Goldschmidt; diquaternary polydimethylsiloxanes, Quaternium-80).

Industrial Applicability

The hair dyes according to the invention can be used as foam, shampoo, cream, gel, lotion, balsam or rinse. Preference is, however, given to use as foam formulation. They typically have the following composition:

a) 0.01-5% m/m, preferably 0.05-2% m/m, in particular 0.1-1% m/m, of cationic biopolymers,

b) 0.05-10% m/m, preferably 0.1-4% m/m, in particular 0.2-2% m/m, of alkyl and/or alkenyl oligoglycosides,

c) 0.01-10% m/m, preferably 0.05-5% m/m, in particular 0.1-1% m/m, of dyes

d) 0-10% m/m, preferably 0.5-5% m/m, in particular 1-3% m/m, of cationic surfactants

with the proviso that the quantitative data optionally with water and with other customary auxiliaries and additives adds up to 100% m/m.

As further auxiliaries and additives, the preparations according to the invention may comprise surfactants, emulsifiers, superfatting agent, thickeners, polymers, silicone compounds, biogenic active ingredients, film formers, preservatives and fragrances.

Surfactants

In addition to the cationic surfactants mentioned as optional component, further surface-active substances which may be present are anionic, nonionic and/or amphoteric or amphoteric surfactants, the proportion of which in the compositions is usually about 1 to 70% by weight, preferably 5 to 50% by weight and in particular 10 to 30% by weight. Typical examples of anionic surfactants are soaps, alkylbenzenesulfonates, alkanesulfonates, olefinsulfonates, alkyl ether sulfonates, glycerol ether sulfonates, α-methyl ester sulfonates, sulfo fatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids, such as, for example, acyl lactylates, acyl tartrates, acyl glutamates and acyl aspartates, alkyl oligoglucoside sulfates, protein fatty acid condensates (in particular vegetable products based on wheat) and alkyl (ether) phosphates. If the anionic surfactants contain polyglycol ether chains, these may have a conventional homolog distribution, but preferably have a narrowed homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers or mixed formals, glucoronic acid derivatives, fatty acid N-alkylglucamides, protein hydrolyzates (in particular vegetable products based on wheat), polyol fatty acid esters, sugar esters, sorbitan esters, polysorbates and amine oxides. If the nonionic surfactants contain polyglycol ether chains, these can have a conventional homolog distribution, but preferably have a narrowed homolog distribution. Typical examples of amphoteric or zwitterionic surfactants are alkylbetaines, alkylamidobetaines, aminopropionates, aminoglycinates, imidazoliniumbetaines and sulfobetaines. Said surfactants are exclusively known compounds. With regard to the structure and preparation of these substances, reference is made to the relevant review works, for example J. Falbe (ed.), “Surfactants in Consumer Products”, Springer Verlag, Berlin, 1987, pp. 54-124 or J. Falbe (ed.), “Katalysatoren, Tenside und Mineralöladditive” [Catalysts, surfactants and mineral oil additives], Thieme Verlag, Stuttgart, 1978, pp. 123-217. Typical examples of particularly suitable mild, i.e. particularly skin-compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefinsulfonates, ether carboxylic acids, alkyl oligoglucosides, fatty acid glucamides, alkylamidobetaines, amphoacetals and/or protein fatty acid condensates, the latter preferably being based on wheat proteins.

Emulsifiers

Suitable emulsifiers are, for example, nonionogenic surfactants from at least one of the following groups:

% addition products of from 2 to 30 mol of ethylene oxide and/or 0 to 5 mol of propylene oxide onto linear fatty alcohols having 8 to 22 carbon atoms, onto fatty acids having 12 to 22 carbon atoms, onto alkylphenols having 8 to 15 carbon atoms in the alkyl group, and alkylamines having 8 to 22 carbon atoms in the alkyl radical;

% alkyl and/or alkenyl oligoglycosides having 8 to 22 carbon atoms in the alk(en)yl radical and ethoxylated analogs thereof;

% addition products of from 1 to 15 mol of ethylene oxide onto castor oil and/or hydrogenated castor oil;

% addition products of from 15 to 60 mol of ethylene oxide onto castor oil and/or hydrogenated castor oil;

% partial esters of glycerol and/or sorbitan with unsaturated, linear or saturated, branched fatty acids having 12 to 22 carbon atoms and/or hydroxycarboxylic acids having 3 to 18 carbon atoms, and adducts thereof having 1 to 30 mol of ethylene oxide;

% partial esters of polyglycerol (average degree of self-condensation 2 to 8), polyethylene glycol (molecular weight 400 to 5000), trimethylolpropane, pentaerythritol, sugar alcohols (e.g. sorbitol), alkyl glucosides (e.g. methyl glucoside, butyl glucoside, lauryl glucoside), and polyglucosides (e.g. cellulose) with saturated and/or unsaturated, linear or branched fatty acids having 12 to 22 carbon atoms and/or hydroxycarboxylic acids having 3 to 18 carbon atoms, and adducts thereof with 1 to 30 mol of ethylene oxide;

% mixed esters of pentaerythritol, fatty acids, citric acid and fatty alcohol according to German patent DE 1165574 and/or mixed esters of fatty acids having 6 to 22 carbon atoms, methylglucose and polyols, preferably glycerol or polyglycerol.

% Mono-, di- and trialkylphosphates and mono-, di- and/or tri-PEG alkylphosphates and salts thereof;

% woolwax alcohols;

% polysiloxane-polyalkyl-polyether copolymers and corresponding derivatives;

% block copolymers e.g. polyethylene glycol-30 dipolyhydroxystearate;

% polymer emulsifiers, e.g. pemulen grades (TR-1, TR-2) from Goodrich;

% polyalkylene glycols and

% glycerol carbonate.

The addition products of ethylene oxide and/or of propylene oxide onto fatty alcohols, fatty acids, alkylphenols or onto castor oil are known, commercially available products. These are homolog mixtures whose average degree of alkoxylation corresponds to the ratio of the quantitative amounts of ethylene oxide and/or propylene oxide and substrate with which the addition reaction is carried out. C_{12/18}-fatty acid mono- and diesters of addition products of ethylene oxide onto glycerol are known from German patent DE 2024051 as refatting agents for cosmetic preparations.

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric diglyceride, malic acid monoglyceride, malic acid diglyceride and technical-grade mixtures thereof which may also comprise small amounts of triglyceride as a minor product from the preparation process. Likewise suitable are addition products of from 1 to 30 mol, preferably 5 to 10 mol, of ethylene oxide onto said partial glycerides.

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate, and technical-grade mixtures thereof. Likewise suitable are addition products of from 1 to 30 mol, preferably 5 to 10 mol of ethylene oxide onto said sorbitan esters.

Typical examples of suitable polyglycerol esters are polyglycerol-2 dipolyhydroxystearate (Dehymuls® PGPH), polyglycerol-3 diisostearate (Lameform® TGI) , poly-glycerol-4 isostearate (Isolan® GI 34), polyglyceryl-3 oleate, diisostearoyl polyglyceryl-3 diisostearate (Isolan® PDI), polyglyceryl-3 methylglucose distearate (Tego Care® 450), polyglyceryl-3 beeswax (Cera Bellina®), polyglyceryl-4 caprate (Polyglycerol Caprate T2010/90), polyglyceryl-3 cetyl ether (Chimexane® NL), polyglyceryl-3 distearate (Cremophor® GS 32) and polyglyceryl polyricinoleate (Admul® WOL 1403) polyglyceryl dimerate isostearate and mixtures thereof. Examples of further suitable polyol esters are the mono-, di- and triesters, optionally reacted with 1 to 30 mol of ethylene oxide, of trimethylolpropane or pentaerythritol with lauric acid, coconut fatty acid, tallow fatty acid, palmitic acid, stearic acid, oleic acid, behenic acid and the like.

Furthermore, zwitterionic surfactants may be used as emulsifiers. Zwitterionic surfactants is the term used to describe those surface-active compounds which carry at least one quaternary ammonium group and at least one carboxylate and a sulfonate group in the molecule.

Particularly suitable zwitterionic surfactants are the so-called betaines, such as N-alkyl-N,N-dimethyl-ammonium glycinates, for example cocoalkyldimethyl-ammonium glycinate, N-acylaminopropyl-N,N-dimethyl-ammonium glycinates, for example cocoacylaminopropyl-dimethylammonium glycinate, and 2-alkyl-3-carboxy-methyl-3-hydroxyethylimidazolines having in each case 8 to 18 carbon atoms in the alkyl or acyl group, and cocoacylaminoethyl hydroxyethylcarboxymethylglycinate. Particular preference is given to the fatty acid amide derivative known under the CTFA name Cocamidopropyl Betaine. Likewise suitable emulsifiers are ampholytic surfactants. Ampholytic surfactants are to be understood as meaning those surface-active compounds which, apart from a C_{8/18}-alkyl or -acyl group in the molecule, contain at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkylimino-dipropionic acids, N-hydroxyethyl-N-alkylamidopropyl-glycines, N-alkyltaurines, N-alkylsarcosines, 2-alkyl-aminopropionic acids and alkylaminoacetic acids having in each case about 8 to 18 carbon atoms in the alkyl group. Particularly preferred ampholytic surfactants are N-cocoalkylaminopropionate, cocoacylaminoethyl-aminopropionate and C_{12/18}-acylsarcosine. Finally, cationic surfactants are also suitable as emulsifiers, in which case those of the ester quat type, preferably methyl-quaternized difatty acid triethanolamine ester salts, are particularly preferred.

Superfatting Agents

Superfatting agents which can be used are substances such as, for example, lanolin and lecithin, and polyethoxylated or acylated lanolin and lecithin derivatives, polyol fatty acid esters, monoglycerides and fatty acid alkanolamides, the latter also serving as foam stabilizers.

Thickeners

Suitable thickeners are, for example, polysaccharides, in particular xanthan gum, guar guar, agar agar, alginates and tyloses, carboxymethylcellulose and hydroxyethylcellulose, and also relatively high molecular weight polyethylene glycol mono- and diesters of fatty acids, polyacrylates, polyvinyl alcohol and polyvinylpyrrolidone, surfactants, such as, for example, fatty alcohol ethoxylates with a narrowed homolog distribution or alkyl oligoglucosides, and electrolytes such as sodium chloride and ammonium chloride.

Polymers

Suitable polymers are, for example, copolymers of diallylammonium salts and acrylamides, quaternized vinylpyrrolidone/vinylamidazole polymers, such as, for example, Luviquat® (BASF), condensation products of polyglycols and amines, quaternized collagen polypeptides, such as, for example, lauryldimonium hydroxypropyl hydrolyzed collagen (Lamequat®L/Grunau), quaternized wheat polypeptides, polyethyleneimine, cationic silicone polymers, such as, for example, amodimethicones, copolymers of adipic acid and dimethylaminohydroxypropyldiethylenetriamine (Cartaretins®/Sandoz), copolymers of acrylic acid with dimethyldiallylammonium chloride (Merquat® 550/Chemviron), polyaminopolyamides, as described, for example, in FR 2252840 A, and their crosslinked water-soluble polymers, condensation products of dihaloalkyls, such as, for example, dibromobutane with bisdialkylamines, such as, for example, bisdimethylamino-1,3-propane, cationic guar gum, such as, for example, Jaguar® CBS, Jaguar® C-17, Jaguar® C-16 from Celanese, quaternized ammonium salt polymers, such as, for example, Mirapol® A-15, Mirapol® AD-1, Mirapol® AZ-1 from Miranol.

Suitable anionic, zwitterionic, amphoteric and nonionic polymers are, for example, vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and esters thereof, polyacrylic acids which are uncrosslinked or crosslinked with polyols, acrylamidopropyltrimethylammonium chloride/acrylate copolymers, octylacrylamide/methyl methacrylate/tertbutylaminoethyl methacrylate/2-hydroxypropyl meth-acrylate copolymers, polyvinylpyrrolidone, vinylpyrrolidone/vinyl acetate copolymers, vinylpyrrolidone/dimethylaminoethyl methacrylate/vinylcaprolactam terpolymers, and optionally derivatized cellulose ethers and silicones. Further suitable polymers and thickeners are listed in Cosmetics & Toiletries Vol. 108, May 1993, page 95ff.

Silicone Compounds

Suitable silicone compounds are, for example, dimethylpolysiloxanes, methylphenylpolysiloxanes, cyclic silicones, and amino-, fatty-acid-, alcohol-, polyether-, epoxy-, fluorine-, glycoside- and/alkyl-modified silicone compounds, which can either be liquid or in resin form at room temperature. Also suitable are simethicones, which are mixtures of dimethicones having an average chain length of from 200 to 300 dimethylsiloxane units and hydrogenated silicates. A detailed review of suitable volatile silicones can additionally be found in Todd et al. in Cosm. Toil. 91, 27 (1976).

Biogenic Active Ingredients

Biogenic active ingredients are to be understood as meaning, for example, amino acids, protein hydrolyzates, ceramides, pseudoceramides, essential oils, plant extracts and vitamin complexes.

Film Formers

Further customary film formers are, for example, polyvinylpyrrolidone, vinylpyrrolidone-vinyl acetate copolymers, polymers of the acrylic acid series, quaternary cellulose derivatives, collagen, hyaluronic acid or salts thereof and similar compounds, copolymers of diallylammonium salts and acrylamides, quaternized vinylpyrrolidone/vinylimidazole polymers, such as, for example, Luviquat (BASF AG, Ludwigshafen/FRG), condensation products of polyglycols and amines, quaternized collagen polypeptides, such as, for example, lauryldimonium hydroxypropyl hydrolyzed collagen (Lamequat L, Grüanau GmbH), polyethyleneimine, cationic silicone polymers, such as, for example, amidomethicones or Dow Corning, Dow Corning Co./US, copolymers of adipic acid and dimethylaminohydroxy-propyldiethylenetriamine (Cartaretins, Sandoz/CH), polyaminopolyamides, as described, for example, in FR-A 22 52 840, and crosslinked water-soluble polymers thereof, cationic guar gum, such as, for example, Jaguar CBS, Jaguar C-17, Jaguar C-16 from Celanese/US, quaternized ammonium salt polymers, such as, for example, Mirapol A15, Mirapol AD-1, Mirapol AZ-1 from Mirano/US.

Preservatives

Suitable preservatives are, for example, phenoxyethanol, formaldehyde solution, parabens, pentanediol or sorbic acid, and the further classes of substance listed in Annex 6, Part A and B of the Cosmetics Directive.

Perfume Oils and Fragrances

Perfume oils which may be mentioned are mixtures of natural and synthetic fragrances. Natural fragrances are extracts from flowers (lily, lavender, rose, jasmine, neroli, ylang-ylang), stems and leaves (geranium, patchouli, petitgrain), fruits (aniseed, coriander, cumin, juniper), fruit peels (bergamot, lemon, orange), roots (mace, angelica, celery, cardamom, costus, iris, calmus), woods (pine wood, sandalwood, guaiac wood, cedar wood, rose wood), herbs and grasses (taragon, lemongrass, sage, thyme), needles and branches (spruce, fir, pine, dwarf-pine), resins and balsams (galbanum, elemi, benzoin, myrrh, olibanum, opoponax). Also suitable are animal raw materials, such as, for example, civet and castoreum. Typical synthetic fragrance compounds are products of the ester, ether, aldehyde, ketone, alcohol and hydrocarbon type. Fragrance compounds of the ester type are, for example, benzyl acetate, phenoxyethyl isobutyrate, p-tertbutylcyclohexyl acetate, linalyl acetate, dimethylbenzylcarbinyl acetate, phenylethyl acetate, linalyl benzoate, benzyl formate, ethylmethylphenyl glycinate, allyl cyclohexylpropionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamen aldehyde, hydroxycitronellal, lilial and bourgeonal, and the ketones include, for example, the ionones, α-isomethylionone and methyl cedryl ketone, the alcohols include anethole, citronellol, eugenol, isoeugenol, geraniol, linalool, phenylethyl alcohol and terpineol, and the hydrocarbons include predominantly the terpenes and balsams. Preference is, however, given to using mixtures of different fragrances which together produce a pleasing fragrance note. Essential oils of relatively low volatility, which are mostly used as aroma components, are also suitable as perfume oils, e.g. sage oil, camomile oil, oil of cloves, melissa oil, mint oil, cinammon leaf oil, linden blossom oil, juniperberry oil, vetiver oil, olibanum oil, galbanum oil, labolanum oil and lavandin oil. Preference is given to using bergamot oil, dihydromyrcenol, lilial, lyral, citronellol, phenylethyl alcohol, α-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix coeur, iso-E-Super, Fixolide NP, evernyl, iraldein gamma, phenylacetic acid, geranyl acetate, benzyl acetate, rose oxide, romillat, irotyl and floramat, alone or in mixtures.

The total amount of auxiliaries and additives may be 1 to 50% by weight, preferably 5 to 40% by weight, based on the compositions.

### EXAMPLES

TABLE 1
Mousse application - quantitative data in %
by weight -

| | | Amount (% |
|---|---|---|
| Composition INCI name | Trade name | by weight |
| Chitosan | Hydagen HCMF | 0.5 |
| Glycolic acid | Glycolic acid | 0.28 |
| Cetyltrimethylammonium | Dehyquart A | 1.0 |
| chloride | | |
| Decyl Glucoside | Plantacare 2000 | 1 |
| Amine Oxide | Standamox PL | 0.3 |
| Hydrolyzed Wheat | Gluadin W40 | 0.5 |
| Proteins | | |
| Cationic dye | Arianor Straw | 0.2 |
| | Yellow | |
| Preservative | | q.s. |
| Water | | ad 100.0 |

The dye was dissolved at room temperature in some of the water and added to the remaining water following the addition and dissolution of all substances in the given order.

TABLE 2
Conditioner application - quantitative data
in % by weight -

| | | Amount (% |
|---|---|---|
| Composition INCI name | Trade name | by weight |
| Chitosan | Hydagen HCMF | 0.2 |
| Glycolic acid 70% | Glycolic acid | 0.12 |
| Cetearyl Alcohol Dipalmitoyl- | Dehyquart C4046 | 7.0 |
| ethyl Hydroxyethylammonium | | |
| Methosulfate Ceteareth-20 | | |
| Cetyltrimethylammonium chloride | Dehyquart A | 1.0 |
| Lauryl Glucoside | Plantacare 1200 | 0.2 |
| Cetylstearyl Alcohol | Lanette O | 2.1 |
| Dye | Arianor Madder | 0.2 |
| | Red | |
| Preservative | | q.s |
| Water | | ad 100.0 |

The fat- and the water-soluble fractions of the formulation were heated to 80-85° C., combined and slowly cold-stirred. The dye was dissolved at room temperature in some of the water and added to the cold-stirred emulsion.

TABLE 3
Gel application - quantitative data in % by
weight -

| | | Amount (% |
|---|---|---|
| Composition INCI name | Trade name | by weight) |
| Part I | | |
| Chitosan | Hydagen HCMF | 0.5 |
| Glycolic Acid 70% | Glycolic acid | 0.28 |
| | 70% | |
| Water | | 37.0 |
| Part II | | |
| Hydroxyethylcellulose | Tylose H 100000 | 1.0 |
| | YP 2 | |
| Triethanolamine | | q.s. |
| Glycolic Acid 70% | Glycolic acid | q.s. |
| | 70% | |
| Preservative | | q.s. |
| Water | | 50 |
| Part III | | |
| Cetyltrimethylammonium | Dehyquart A | 1.0 |
| chloride | | |
| Lauryl Glucoside | Plantacare 1200 | 0.2 |
| Part IV | | |
| Cationic dye | Arianor Straw | 0.2 |
| | yellow | |
| Water | | ad 100.0 |

Part I was prepared by dispersing chitosan and glycolic acid in water.

Part II was prepared separately by dispersing Tylose in the preserved triethanolamine-containing water, where, after the Tylose had been swelled with glycolic acid, neutralization was carried out to a pH of pH 4.5.

Then, Parts I and II were mixed and then Part III was added. Finally, the cationic dye was dissolved in the remaining water and distributed homogeneously within the gel.

1. A hair dye containing:
A) cationic biopolymers,
B) alkyl and/or alkenyl oligoglycosides and
C) dyes:

2. The hair dye as claimed in claim 1, characterized in that it comprises, as cationic biopolymers (A), chitosans and/or chitosan derivatives.

3. The hair dye as claimed in claims 1 and/or 2, characterized in that it comprises, as component (B), alkyl and/or alkenyl oligoglycosides of the formula (I),
R¹O—[G]ₚ (I)
in which R¹ is an alkyl and/or alkenyl radical having 4 to 22 carbon atoms, G is a sugar radical having 5 or 6 carbon atoms and p is a number from 1 to 10.

4. The hair dye as claimed in at least one of claims 1 to 3, characterized in that it comprises, as component (C), oxidation dyes and/or direct dyes.

5. The hair dye as claimed in at least one of claims 1 to 4, characterized in that it comprises cationic surfactants as optional component (D).

6. The hair dye as claimed in at least one of claims 1 to 5, characterized in that the cationic biopolymers are present in an amount of from 0.01 to 5% by weight.

7. The hair dye as claimed in at least one of claims 1 to 6, characterized in that alkyl and/or alkenyl oligoglycosides are present in an amount of from 0.1 to 10% by weight.

8. The hair dye as claimed in at least one of claims 1 to 7, characterized in that dyes are present in an amount of from 0.01 to 10% by weight.

9. The hair dye as claimed in at least one of claims 1 to 8, characterized in that it is in the form of a foam.

10. The use of mixtures containing:
A) cationic biopolymers,
B) alkyl and/or alkenyl oligoglycosides and
C) dyes
for dyeing keratin fibers.
